(19) United States
(12) Patent Application Publication
(10) Pub. No.: US 2004/0101572 A1
(43) Pub. Date: May 27, 2004

(54) Microbial control system

(76) Inventors: Bryan E. Kepner, Atlanta, GA (US); Sherman M. Ponder, Norcross, GA (US)

Correspondence Address: John A. Parrish, Law Offices of John A. Parrish, Suite 300, Two Bala Plaza, Bala Cynwyd, PA, 19004, US

(21) Appl. No.: 10/383,168

(22) Filed: Mar. 5, 2003

Related U.S. Application Data
(60) Provisional application No. 60/361,997, filed on Mar. 6, 2002.

(51) Int. Cl. A61K 33/24; A61K 33/38; A61K 33/34

(52) U.S. Cl. 424/617; 424/618; 424/630

(57) ABSTRACT

Te invention relates to a microbial control system for treating influent water and sump water for control of microbial material in machines which process water such as ice making machines, humidifiers such as cool mist humidifiers and cooling towers. The microbial control system includes antimicrobial treatment media housed in a containment vessel. The treatment media can include any one or more of transition metals and transition metal oxides. The transition metal may be any of Sc, Ti, V, Cr, Mn, Fe, Co, Ni, Cu, Zn, Y, Zr, Nb, Mo, Tc, Ru, Rh, Pd, Ag, Cd, Hf, Ta, W, Re, Os, Ir, Pt, Au, Hg, Rf, Db, Sg, Bh, Hs, Mt, Uun, Uuu and Uub.

This application claims the benefit of priority to U.S. Provisional application No. 60/361,997 filed Mar. 6, 2002.

### FIELD OF THE INVENTION

The invention relates to a system for control of microbial growth in water, especially in water employed in ice manufacture and in humidification.

### BACKGROUND OF THE INVENTION

Control of microbial growth is important in devices where water is processed. An amount of water, chlorinated or not, that is allowed to accumulate and stand tends to foster microbial growth. Solid surfaces of devices which are continually and/or sporadically wetted also foster microbial growth. This growth can occur from both the (non-pathogenic) bacteria present in treated water, as well as opportunistic air-borne bacteria, yeasts, and molds in the water per se or the wetted surfaces.

Municipal water is typically treated by chlorination to reduce bacteria and other microorganisms. Chlorination does not, however, kill all of the bacteria present in the water. Also, chlorination does not control water purity in terms of total dissolved solids (TDS) or aqueous metal content.

Control of microbial growth is very important in devices such as commercial and residential ice machines, as well as room humidifiers vaporizers and cooling towers. Most ice machines use a sump in the form of a small (typically 1-5 gallon capacity) open tank that receives influent water. The water in the sump is chilled and is circulated by a pump to ice-forming racks to cascade down the surfaces of the racks. The ice forming racks are held at low temperature during the ice-making cycle to accrete ice as the sump water passes over their outer surfaces to form ice cubes.

The ice forming racks contain numerous indentations and bumps. Strictly laminar gravitational flow of the sump water down these racks therefore is not possible. As a result, considerable amount of splash water is generated within the ice-making machine. Microbes present in the splash water, as well as opportunistic air-borne organisms, are conveyed by the splash water to the interior splash zone surfaces of the ice machine. Subsequent splashing onto the splash zone surfaces as the ice-making cycle continues provides regular re-wetting and aeration of the microorganisms. This splashing forms droplets which are caught in the sump for re-entry into the ice-making cycle. These droplets can entrain bacteria and mold colonies present on the splash zone surfaces, and thereby re-infest the sump water.

The forgoing is thought to be a principle reason for the failure of the internal cleaning systems of ice machines. Typically, these cleaning systems treat the influent water with, e.g., benzalkonium chloride, to kill the vast majority of organisms entering the ice-making machine. These cleaning systems, however, do not kill 100% of all influent organisms, nor do they treat the splash zone surfaces. Thus, a single microorganism has the potential to be splashed onto an interior splash zone surface where continuous watering and aeration is conducive to growth. This single microorganism, as it multiplies and is recirculated throughout the ice machine, can consequently cause infestation of all of the interior splash zone surfaces, as well as the sump water and ice.

Room humidifiers such as portable mist type humidifiers also are susceptible to bacteria and fungi growth within their water reservoirs. This bacteria and fungi can be transmitted into the air though the “misting” or atomization of water by the humidifier. This can cause significant health concerns for children, elderly, or anyone who has a weakened immune system.

It is known that chemical additives in the plastic components of some humidifiers can control fungi that may grow on the plastic surfaces of the humidifier. However, since these additives are found only in the plastic components of the humidifier, they offer little or no protection from the growth of bacteria or fungi in the water present in the humidifier where there is the most concern for transmission into the surrounding air.

Some humidifiers employ replaceable air filters to minimize bacteria emission into the air. The majority of bacteria and fungi in humidifiers, however, is derived from the water per se since chlorinated tap water contains low levels of Heterotrophic Plate Count bacteria. These bacteria typically are present in amounts sufficient to propagate within the humidifier tank. Air filtration therefore offers little or no protection from growth of this type of bacteria. Use of bottled, well derived, filtered, or distilled water instead of tap water in a room humidifier can cause even greater risks. This is because these sources of water do not contain residual chlorine or other disinfection agents and thus frequently have extremely high concentrations of bacteria.

Because of the health risks associated with microbiological growth, such as, bacterial and fungi, a need exists for a system for control of microbial growth in devices which process water. In particular, a need exists for system for control of microbial growth in devices such as ice making machines and humidifiers, as well as for control of microbial growth in sumps, holding tanks, dehumidifiers, tea and coffee makers, water filtration devices, air conditioners and air conditioning systems, water pitchers, water tanks, ballast tanks, swimminng pools, spas, and cooling towers.

### BRIEF DESCRIPTION OF THE DRAWINGS

FIG. 1 is a side view of a containment vessel used in the microbial control system of the invention.

FIG. 1A is a top view of the containment vessel of FIG. 1.

FIG. 2 is a side view of a cap for the containment vessel of FIG. 1.

FIG. 2A is a top view of the cap of FIG. 2.

FIG. 2B is a end view of the cap of FIG. 2

FIG. 3 is an exploded view of the alternative embodiment of a container vessel for use in the microbial control system.

FIG. 4 is an assembly view of the container vessel shown in FIG. 3.

FIG. 5 is a partial exploded view of the container vessel of FIGS. 3 and 4 showing the presence of antimicrobial treatment material in the vessel

FIG. 6 is a bottom view of the hanger cap shown in FIG. 3.

FIG. 7 is a side view of the hanger cap shown in FIG. 3.

### SUMMARY OF THE INVENTION

In a first aspect, the invention relates to a microbial control system for treating influent water and sump water for control of microbial material such as bacteria in machines which process water. In particular, the invention relates to microbial control systems for use in ice making machines. Another aspect of the invention relates to microbial control systems for control of bacteria and fungi in humidifiers such as cool mist humidifiers.

The microbial control system includes antimicrobial treatment media housed in a containment vessel. The treatment media can include any one or more of Sn as well as transition metals and transition metal oxides. The treatment media can be included on an inert support material and may be in the form of any one of solid particles or layers on the support material. The transition metal may be any of Sc, Ti, V, Cr, Mn, Fe, Co, Ni, Cu, Zn, Y, Zr, Nb, Mo, Tc, Ru, Rh, Pd, Ag, Cd, Hf, Ta, W, Re, Os, Ir, Pt, Au, Hg, Rf, Db, Sg, Bh, Hs, Mt, Uun, Uuu and Uub, preferably Ag, Cu and Zn. The transition metal also may be transition metal alloy such as CuZn. The oxide preferably is an oxide of any one of Ag, Cu, Zn and Sn, more preferably an oxide of any one of Ag and Cu. The support material may be any of activated carbon, alumina, silica, titanium oxide, tin oxide, lanthanum oxide, copper oxide, vanadium oxide, manganese oxide, nickel oxide, iron oxide, zinc oxide, zirconium oxide, magnesium oxide thorium oxide, polyethylene, polypropylene, polyvinylchloride, polystyrene and polyethylene terephthalate, preferably any of alumina and polyethylene terephthalate. When the transition metal is Ag, the Ag in the microbial control system may provide solvated silver ions at a concentration of about 1 ppb to about 1000 ppb.

The treatment media may have a metal content of about 0.01 wt. % to about 15 wt. %., preferably about 0.35 wt. % to about 3.5 wt. %. In a preferred aspect, the treatment media is a mixture of Ag coated onto alumina and Cu coated on alumina wherein the Ag is present in an amount 0.7% Ag based on total weight of Ag and alumina and Cu is present in an amount of 4.0% Cu based on total weight of Cu and alumina. In another aspect, the treatment media are mixtures of nanoparticles of Ag and Cu wherein each of the Ag and Cu have a size of about 0.1 nm to about 10,000 nm. Preferably, the treatment media is a mixture of nanoparticles of Ag and Cu wherein each of the Ag and Cu have a size of about 2 nm to about 500 nm and wherein the ratio of Ag to Cu in the mixture is about 1:1. In yet another aspect, the treatment media is a mixture of nanoparticles of silver and copper on alumina and the silver nanoparticles have a median size of about 20 nm and the copper nanoparticles have a median size of about 100 nm. In this aspect, each of the silver nanoparticles and the copper nanoparticles are present in the mixture in an amount of about 0.2 wt. % to about 4.8 wt. % based on the total combined weight of the metal and the alumina support material and the silver nanoparticles and the copper nanoparticles are present in the mixture in a ratio of 1:5. In yet a further aspect, the treatment media comprises a mixture of silver oxide and copper oxide on alumna support material. In this aspect, the silver oxide may be present in the mixture in an amount of about 0.1 wt. % to about 2 wt. %, remainder copper oxide.

The treatment media also may be a mixture of nanoparticles of silver and copper in combination with nanoparticles of any one of additive metals or additive oxides In this aspect, the mixture of nanoparticles of silver and copper may be employed in combination with nanoparticles of any one of additive metals or additive oxides. The additive metals may be any of Sc, Ti, V, Sn, Cr, Mn, Fe, Co, Ni, Zn, Y, Zr, Nb, Mo, Tc, Ru, Rh, Pd, Cd, Hf, Ta, W, Re, Os, Ir, Pt, Au, Hg, Rf, Db, Sg, Bh, Hs, Mt, Uun, Uuu and Uub. The additive metal oxides may be any of alumina, silica, silver oxide, titanium oxide, tin oxide, lanthanum oxide, copper oxide, vanadium oxide, manganese oxide, nickel oxide, iron oxide, zinc oxide, zirconium oxide, magnesium oxide, thorium oxide.

In a further embodiment, the invention relates to an ice making machine that employs the a microbial growth control system for control of microbial growth in any of influent water and sump water processed by the ice making machine. The microbial control system, as described above, may include any of transition metals or transition metal oxides, wherein the transition metal is selected from the group consisting of Sc, Ti, V, Cr, Mn, Fe, Co, Ni, Cu, Zn, Y, Zr, Nb, Mo, Tc, Ru, Rh, Pd, Ag, Cd, Hf, Ta, W, Re, Os, Ir, Pt, Au, Hg, Rf, Db, Sg, Bh, Hs, Mt, Uun, Uuu and Uub. Typically, the influent water processed by the ice making machine has a flow rate of more than about one bed volume per minute and the influent water has more than about 5×10⁻⁶ m dissolved oxygen, preferably about 5×10⁻³ m to about 3×10⁻⁴ m. The influent water typically is at temperature of less than about 45 C. In a preferred aspect, the microbial control system employed in the ice making machine includes a 50:50 mixture of component A formed from 2-500 nm thick Ag on 2-3 mm alumina beads and component B formed from 2-500 nm thick Cu on 2-3 mm alumina beads where component A has 0.7% Ag based on total weight of Ag and alumina and component B has 4.0% Cu based on total weight of Cu and alumina. The transition metal oxides which may be employed in the microbial control system of the ice making machine may be an oxide of any one of Ag and Cu.

Another embodiment of the invention relates to a humidifier, such as a mist humidifier, that includes a microbial control system for control of microbial growth in water processed by the humidifier. The microbial control system includes antimicrobial treatment media, and the antimicrobial treatment media may be any of transition metals or transition metal oxides such as Sc, Ti, V, Cr, Mn, Fe, Co, Ni, Cu, Zn, Y, Zr, Nb, Mo, Tc, Ru, Rh, Pd, Ag, Cd, Hf, Ta, W, Re, Os, Ir, Pt, Au, Hg, Rf, Db, Sg, Bh, Hs, Mt, Uun, Uuu and Uub. The humidifier processes influent water that has more than about 5×10⁻⁶ m dissolved oxygen, preferably about 5×10⁻³ m to about 3×10⁻⁴ m, and which has a temperature of less than about 35 C. In a preferred aspect, the microbial control system includes a 50:50 mixture of component A formed from 2-500 nm thick Ag on 2-3 mm alumina beads and component B formed from 2-500 nm thick Cu on 2-3 mm alumina beads. Component A has 0.7% Ag based on total weight of Ag and alumina and component B has 4.0% Cu based on total weight of Cu and alumina. The transition metal oxide employed in the microbial control system of the humidifier may be an oxide of any one of Ag and Cu.

In yet another embodiment, the invention relates to a cooling tower that includes a microbial control system for control of microbial growth in water processed by the cooling tower. The microbial control system includes antimicrobial treatment media which may include any of transition metals or transition metal oxides, wherein the transition metal is selected from the group consisting of Sc, Ti, V, Cr, Mn, Fe, Co, Ni, Cu, Zn, Y, Zr, Nb, Mo, Tc, Ru, Rh, Pd, Ag, Cd, Hf, Ta, W, Re, Os, Ir, Pt, Au, Hg, Rf, Db, Sg, Bh, Hs, Mt, Uun, Uuu and Uub.

In use, microbial control system is placed into an advantageous location of device which processes water, such as within the water circulation system or water storage area of the device, to allow the water to contact antimicrobial media in the vessel so as to release antimicrobial metal into the water. This release may be by due forces of abrasion from the containment vessel of the microbial control system while in an area where water is actively flowing across the vessel. Release also may be caused by Brownian motion only where little. to no flow exists.

Aqueous feedstock can be flowed through the antimicrobial treatment media over a wide range of flow rates. The feedstock also may be flowed over the media by Brownian motion only. Typically, the flow rate is about 0.01-bed volumes/minute to about 20-bed volumes/minute, preferably about 0.1-bed volumes/minute to about 10-bed volumes/minute. The specific flow rate may be varied in accordance with the type and amount of treatment media in the containment vessel, the packing density of the treatment media, the type of water undergoing treatment, such as influent water or sump water, the size of the sump in which the containment vessel is placed, as well as the porosity of the containment vessel.

The microbial control system may be used in any device which processes water. Examples of these devices include ice making machines and humidifiers. Ice making machines where the microbial control system of the invention may be used include but are not limited to cubed, crushed and flaked ice makers, as well as home freezer and commercial bulk ice makers.

The microbial control system also may be employed in a wide variety of other applications where standing water is present. Examples of these applications include but are not limited to sumps, holding tanks, dehumidifiers, tea and coffee makers, water filtration devices, air conditioners and air conditioning systems, water pitchers, water tanks, ballast tanks, swimming pools, spas, and cooling towers.

When employed in ice making machines, the microbial control system achieves antimicrobial and bacteriostatic action, typically constant antimicrobial and bacteriostatic action, in treatment of the influent water and the sump water, as well as the interior splash zone surfaces of those machines. Typically this action is achieved over the entire cycle of ice formation and lasts for the life of the antimicrobial media used in the system. The antimicrobial activity of the system depends on the size of the containment vessel, and the type and amount of treatment media in the vessel, the water volume being treated, and the quality of the water being treated.

When employed in humidifiers such as cool mist humidifiers, the microbial control system achieves constant antimicrobial and bacteriostatic action during the life of the microbial control media within the containment vessel of the microbial control system.

Having summarized the invention, the invention is described in detail below by reference to the following detailed specification and non-limiting examples.

### DETAILED DESCRIPTION OF THE INVENTION

The microbial control system includes antimicrobial treatment media housed in a porous containment vessel. The antimicrobial treatment media includes transition metals and/or transition metal oxides. The treatment media typically are on an inert support material. The treatment media can be in the form of solid particles or layers of one or more zero valent transition metals or metal oxides on a support material. Where layered treatment media are employed, these media may be produced by methods such as plasma spraying, liquid spraying, sputtering, incipient wetness, and gas phase impregnation.

The treatment media are selected from transition metals, transition metal oxides, as well as mixtures thereof from Groups 3-12 of the Periodic Table. Examples of transition metals which may be employed include Sc, Ti, V, Cr, Mn, Fe, Co, Ni, Cu, Zn, Y, Zr, Nb, Mo, Tc, Ru, Rh, Pd, Ag, Cd, Hf, Ta, W, Re, Os, Ir, Pt, Au, Hg, Rf, Db, Sg, Bh, Hs, Mt, Uun, Uuu and Uub, preferably Ag, Cu, Zn, most preferably Ag and Cu. Examples of transition metal oxides include Ag, Cu, Zn and Sn, preferably Ag, Cu and Zn, most preferably Ag and Cu. In addition, alloys of transition metals such as CuZn manufactured by KDF Fluid Treatment, Inc. of Michigan may be employed.

The transition metal may be employed in a wide range of sizes depending on the specific application. When used as layers on a support, the transition metals employed as treatment media are nanoparticles of Ag of about 0.1 nm to about 10,000 nm, preferably about 1 nm to about 1000 nm, more preferably about 2 nm to about 500 nm diameter. Where Ag is employed as the treatment media, the microbial control system provides solvated silver ions at a concentration of about 1 ppb to about 1000 ppb for control of microbial growth within potable water systems However for some applications the levels of solvated Ag ions may be higher as desired.

The transition metal/transition metal oxide treatment media preferably are on a support material to better enable the transition metal/transition metal oxide media to be exposed to the aqueous feedstock. The support material is inert, non-bioactive, and aqueously insoluble. The support material may be porous or non-porous. Useful support materials may include, but not limited to activated carbon, oxides such as alumina and silica, as well as oxides of titanium, tin, lanthanum, copper, vanadium, manganese, nickel, iron, zinc, zirconium, magnesium, thorium, or a combination thereof, preferably alumina. Other useful supports include plastics such as polyethylene, polypropylene, phenolics, and polyvinylchloride, preferably polypropylene, and insoluble resins such as polystyrene and polyethylene terephthalate, preferably polyethylene terephthalate.

The shape of the support material may be regular or irregular, e.g., spherical or pyramidal, over a wide range of sizes. The particle size of spherical support materials may be about 0.001 inches to about 0.5 inches in diameter, preferably about 0.0625 inches to about 0.25 inches in diameter, most preferably about 0.1 inches to about 0.19 inches in diameter.

The metal content of the treatment media may be about 0.01 wt. % to about 15 wt. %, preferably about 0.1 wt. % to about 7.4 wt. %, more preferably about 0.2 wt. % to about 4.8 wt. %, most preferably about 0.35 wt. % to about 3.5 wt. % based on the total weight of the media, including support material. In a preferred aspect, the treatment media is MB2001-B and MB2002-B, each of which are available from Apyron Technologies, Inc. MB 2001-B is 2-500 nm thick Ag coated onto 2-3 mm alumina beads. MB 2001B has 0.7% Ag based on total weight of Ag and alumina. MB 2002-B is 2-500 nm thick Cu coated onto 2-3 mm alumina beads. MB2002B has 4.0% Cu based on total weight of Cu and alumina. Other commercially available materials which may be used as treatment media include but are not limited to silver on zeolite made by Sinanen Co., Ltd., silver, copper, and zinc on spherical supports made by Fountainhead Technologies, Inc., and Silver impregnated Carbon available from Barnaby Sutcliff Corporation.

Preferred treatment media include mixtures of Ag/Cu, Ag/Zn, Ag/Sn and Ag/Ni. More preferably, the treatment media are mixtures of nanoparticles of Ag and Cu each of which have a size of about 0.1 nm to about 10,000 nm, preferably about 1 nm to about 1000 nm, more preferably of about 2 nm to about 500 nm. The ratio of Ag to Cu in the mixtures may vary from about 100:1::Ag:Cu, preferably about 10:1::Ag:Cu to about 5:1, more preferably about 1:1::Ag:Cu:.

In another preferred aspect of the invention, the treatment media includes a mixture of nanoparticles of silver and copper metal on an alumina support material. The silver nanoparticles have a median size of about 20 nm and the copper nanoparticles have a median size of about 100 nm. Each of the silver nanoparticles and the copper nanoparticles may be present in the mixture in an amount of about 0.2 wt. % to about 4.8 wt. %, preferably about 0.5 wt. % to about 4.5 wt. %, more preferably about 0.7 wt. % to about 4.0 wt. % based on the total combined weight of the metal and the alumina support material. In an especially preferred aspect, the media material is a 1:1 mixture of nanoparticle sized Ag and nanoparticle sized Cu on alumina support material.

In another aspect, the treatment media may include a mixture of silver oxide and copper oxide on a support material. Useful copper oxides include both cuprous oxide and cupric oxide, preferably cuprous oxide. The amounts of silver oxide and copper oxide may vary over a wide range. Typically the silver oxide is about 0.1 wt. % to about 2 wt. %, preferably about 0.5 wt. % to about 1.5 wt. %, more preferably about 0.7 wt. % to about 1 wt. % of the mixture, the remainder copper oxide. The purities of silver oxide and copper oxide may vary over a wide range. Typically the oxides are about 80 wt. % to about 99.999% pure, preferably about 90% pure to about 99.99% pure, more preferably about 98% to about 99.99% pure.

In yet another aspect of the invention, the treatment media is a mixture of nanoparticles of silver and copper metal in combination with nanoparticles of one or more additive metals or metal oxides from Groups 2-13 of the Periodic Table. The additive metals may be Sc, Ti, V, Sn, Cr, Mn, Fe, Co, Ni, Zn, Y, Zr, Nb, Mo, Tc, Ru, Rh, Pd, Cd, Hf, Ta, W, Re, Os, Ir, Pt, Au, Hg, Rf, Db, Sg, Bh, Hs, Mt, Uun, Uuu and Uub, more preferably Zn, Sn, Ni, most preferably Zn and Sn. The additive metal oxides may be oxides such as alumina and silica, as well as oxides of silver, titanium, tin, lanthanum, copper, vanadium, manganese, nickel, iron, zinc, zirconium, magnesium, thorium, or a combination thereof, preferably silver, copper, tin, zinc, and nickel, more preferably silver. The additive metal or metal oxide may be present in an amount of about 0.01 wt % to about 99.9 wt %, preferably about 0.1 wt % to about 10 wt %, more preferably about 1 wt % to about 5 wt %, based on the weight of the mixture of silver and copper. In this aspect, the combined weight of silver and copper in the mixture is about 0.1 wt. % to about 5 wt. %, and the weight of additive metal or metal oxide is about 0.05 wt. % to about 5 wt. %, all amounts based on the total weight of silver, copper as well as additive metal or metal oxide.

The containment vessel employed in the microbial control system prevents the treatment media from dispersing into the aqueous feedstock which is undergoing treatment while allowing free flow of the aqueous feedstock to contact the treatment media. The containment vessel is formed from an inert, aqueously insoluble material such as acrylonitrile butadiene styrene (ABS), polyvinylchloride (PVC), high density polyethylene (HDPE), polypropylene (PP), low density polyethylene (LDPE), Nylon, Delrin, urethane, vinyl, ultrahigh molecular weight polypropylene (UHMWPP), polyurethane, phenolics, Plexiglas, stainless steel, carbon steel, aluminum or wire mesh, preferably PP and Nylon.

The containment vessel may be formed in a variety of shapes, preferably in the form of a square, round, octagonal, or hexagonal cylinder. Useful containment vessels have more than about 10% porosity, preferably more than about 20% porosity, most preferably more than about 25% porosity. The pore spaces of the containment vessel, in order to retain the treatment media, typically are less than about 0.6 to about 0.75 times the smallest average diameter of the enclosed treatment media. The pore spaces of the containment vessel may be in the form of slots or holes, or a combination of both, provided the dimensions of the pore spaces are as described above. The interior volume of the containment vessel may vary depending on the application in which the containment vessel is used. Containment vessels for use in ice making machines typically have interior volumes of about 25 cc to about 150 cc. Containment vessels for use in applications such as humidifiers typically have interior volumes of about 10 cc to about 100 cc. The size of the containment vessel, as well as quantity and type of antimicrobial treatment media may be varied over a wide range. Typically, the treatment media in the containment vessel has a packing density of about 70% to about 90%,

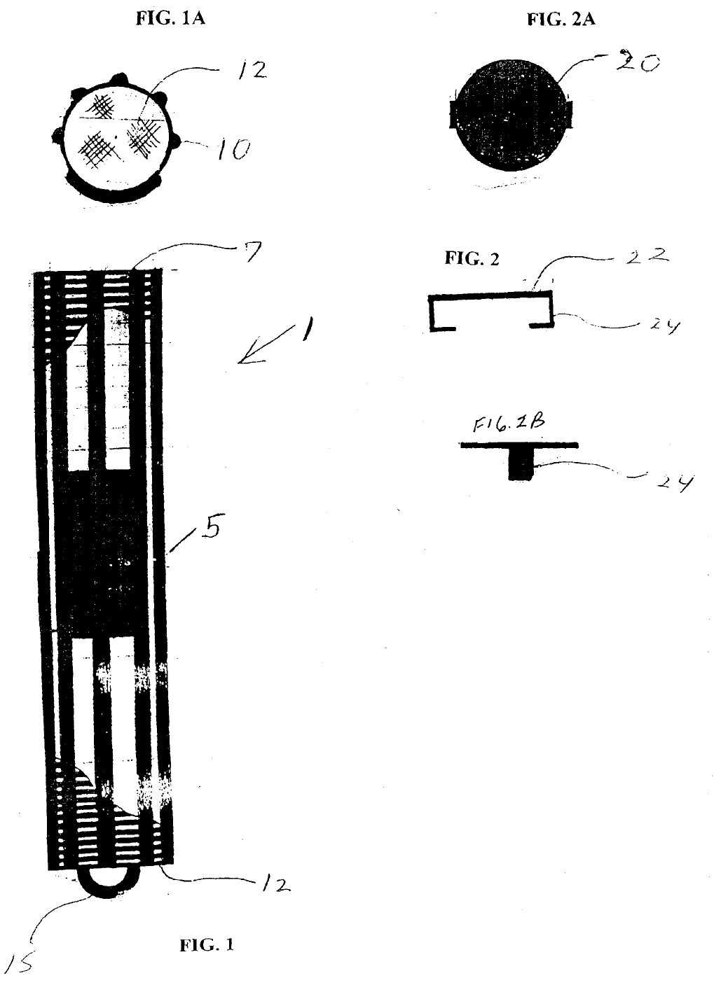

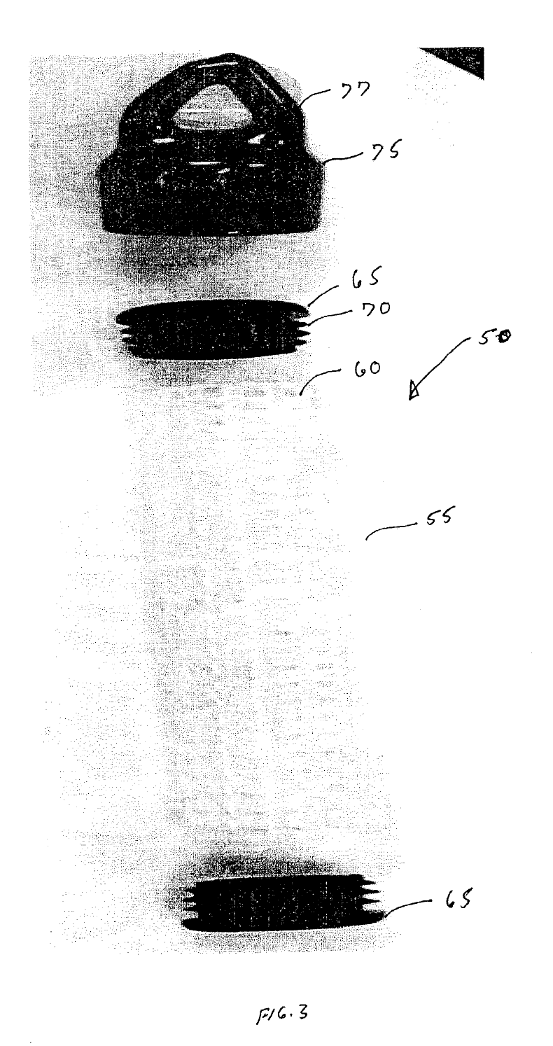

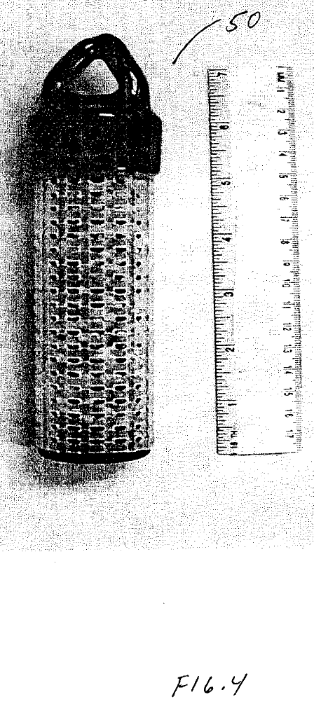

An embodiment of a containment vessel of the antimicrobial control system for use in an ice making machine is shown in FIGS. 1 and 2. In this embodiment, all components are formed of a plastic such as ABS or PVC. As shown in FIGS. 1 and 2, containment vessel 1 includes slotted circular cylinder 5 that is integrally joined to solid bottom plate 12. Cap 20 is releasably secured to the top of cylinder 5. Loop 15 can be attached to the bottom of plate 12 to facilitate handling of vessel 1. Cylinder 5 can include a plurality of longitudinal reinforcing ribs 10 which preferably are uniformly spaced around the circumference of cylinder 5. Cylinder 5 has open slots 7 spaced along the length of cylinder 5. Slots 7 typically have a width and spacing of up to about 0.6 to about 0.7 times the diameter of the supported treatment media in containment vessel 1. Cap 20, as shown in FIGS. 2-2A, can be in the form of a cylindrical plate 22 that has downwardly facing locking tabs 24. Tabs 24 engage slots 7 to releasably secure cap 20 to cylinder 5.

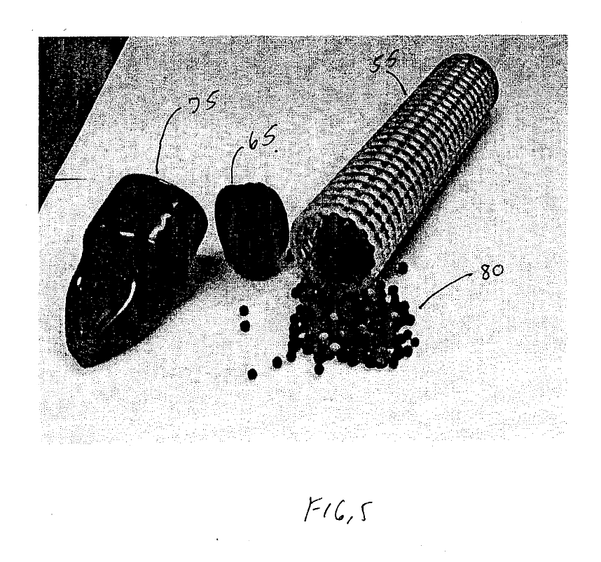

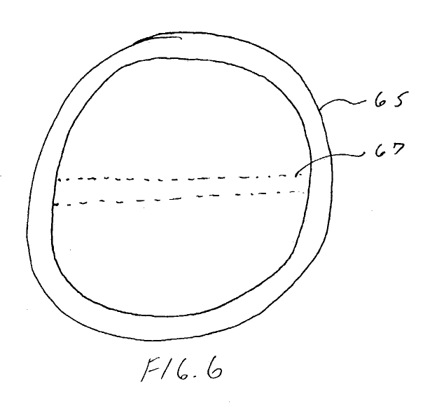

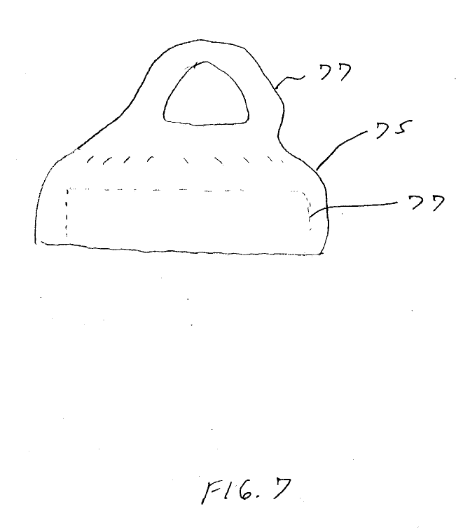

In an alternative embodiment of the container vessel of the microbial control system for use devices such as ice making machines, and humidifiers is shown in FIGS. 3-5. As shown, containment vessel 50 includes a porous tubular member 55 which has slots 60 therein. Although slots 60 shown in container vessel 55 are rectangular, it is to be understood that there is no such limitation as to the configuration of slots 60. Treatment media 80, as shown in FIG. 5, is included in containment vessel 50. Tubular member 55 may be made of polypropylene. End caps 65 are provided for insertion into the open ends of tubular member 55. End caps 65 can be made of, for example, nylon. End caps 65 include flexible circular ribs 70 which, when inserted into tubular member 55, securely seal end caps 65 to tubular member 55. An optional, hanger cap 75 made of a flexible material such as vinyl may be placed over endcap 65 as shown in FIG. 4. Hanger cap 75, as shown in FIGS. 3, 6 and 7, includes raised portion 77 for ready manipulation of hanger cap 75. Hanger cap 75 includes recess 77 for joining of hanger cap 75 to endcap 65 and tubular member 55. Hanger cap 75 provides a convenient means for carrying assembled containment vessel 50. Containment vessel 50 may also be used in devices such as a humidifier.

The microbial control system can treat the influent and sump water as well as splash zone surfaces with precise dosages of antimicrobial agent in amounts proportional to the rate and amount of microbial infestation. When employed in a device such as an ice making machine, the microbial control system may be positioned in the flow of an aqueous feedstock such as potable water. Typically, the flow rate is greater than about one bed volume per minute. The microbial control system also can be placed in static vessels where only Brownian motion exists.

Aqueous feedstocks useful in ice making machines where the microbial control system is employed typically have more than about 5×10⁻⁶ m dissolved oxygen, preferably about 5×10⁻³ m oxygen to about 3×10⁻⁴ oxygen. The temperature of the feedstock typically is less than about 45° C., preferably less than about 10° C.

Aqueous feedstocks useful in humidifiers where the microbial control system is employed typically have more than about 5×10⁻⁶m dissolved oxygen, preferably about 5×10⁻³m oxygen to about 3×10⁻⁴m oxygen. The temperature of this feedstock typically is about 40° C. to about 20° C., preferably about 35° C.

The invention will now be described by reference to the following non-limiting examples.

### EXAMPLES 1-27

### Ice Making Machines

In examples 1-27, two identical ice making machines, (model no. CME506 from Scostman) each capable of making 500 lb of ice per day, are operated continuously by removing the ice before the bins fill up. Both machines receive influent city tap water at 60 psi. Both machines are fitted with a 20 micron particle filter and a granulated activated carbon (GAC) filter to remove particles and chlorine from the water prior to entry into the ice making machine.

Both machines are initially operated until bacterial counts in the sump average more than about 400 CFU/ml. At that time, a microbial control system that includes 26 gm of MB2001-B and 26 gm of MB2002-B in a 100 cc containment vessel is placed into the sump water recycling area of ice machine #1. The containment vessel is a slotted vessel as shown in FIGS. 1 and 2. The amount of open pores in the containment vessel is 30 percent. The sump water recycling area of the ice making machine has a volume of two gallons. For comparison, the second ice making machine operates without a microbial control system.

Ice samples are taken daily from both machines by collecting the harvested ice between the delivery chute and prior to reaching the ice bin of the machine. The ice samples are allowed to melt at room temperature. The water from the ice is aseptically plated onto sterile petri dishes of R2A agar by the spread-plate method. Additionally, water samples are drawn from the sump area using a sterile collection tube on a wire hanger. Also, influent samples are taken from a sampling port located on the influent line prior to te ice machine but after the GAC filter.

All samples are aseptically plated on to sterile petri dishes of R2A agar by the spread-plate method. Plates are incubated for 7 days at 25° C., and then counted. All forms of microbial growth found, including bacteria, yeasts, and molds, are counted with equal weight. The comparative results of the bacterial counts (in CFU/ml) in the ice produced by machines one and two are presented in Table 1.

TABLE 1

| | | | | Machine 2 - Treated | | | |
|---|---|---|---|---|---|---|---|
| Machine 1 - Untreated | | | | With the Invention | | | |
| Exam- | Bacteria counts CFU/ml | | | Example | Bacteria counts CFU/ml | | |
| ple # | Influent | Sump | Ice | # | Influent | Sump | Ice |
| 1 | 300 | 476 | 416 | 1a | 7300 | 1 | 52 |
| 2 | 150 | 564 | 1095 | 2a | 4900 | 1 | 648 |
| 3 | 200 | 690 | 770 | 3a | 9600 | I | 87 |
| 4 | 100 | 614 | 1740 | 4a | 6150 | 1 | 7 |
| 5 | 306 | 360 | 360 | 5a | 6700 | 1 | 11 |
| 6 | 350 | 288 | 420 | 6a | 4500 | 1 | 2 |
| 7 | 194 | 550 | 550 | 7a | 8050 | 2 | 1 |
| 8 | 1250 | 372 | 440 | 8a | 5350 | 1 | 19 |
| 9 | 5650 | 404 | 710 | 9a | 18950 | 1 | 2 |
| 10 | 4224 | 300 | 475 | 10a | 6900 | 1 | 2 |
| 11 | — | 450 | 890 | 11a | 3700 | 1 | 3 |
| | | | | 11b | 2700 | — | 7 |
| 12 | 684 | 700 | 1025 | 12a | 3300 | 1 | 11 |
| 13 | | 1850 | 1250 | 13a | 4700 | 1 | 5 |
| 14 | 2050 | 1050 | 885 | 14a | 21100 | 1 | 1 |
| IS | 2800 | 575 | 1260 | 15a | 8300 | 2 | 42 |
| 16 | 1800 | 430 | 510 | 16a | 7600 | 1 | 1 |
| 17 | 2050 | 335 | 600 | 17a | 4050 | 1 | 2 |
| | | | | 17b | — | — | — |
| 18 | 6800 | 3200 | 2240 | 18a | 4550 | 1 | 2 |
| 19 | 1800 | 420 | 1275 | 19a | 3150 | 1 | 7 |
| 20 | 5600 | 170 | 340 | 20a | 5400 | 1 | 17 |
| 21 | 3000 | 665 | 1470 | 21a | 1075 | 1 | 98 |
| 22 | 2400 | 500 | 960 | 22a | 5000 | 1 | 1 |
| 23 | 7750 | 1510 | 1530 | 23a | 4000 | 1 | 3 |
| 24 | 13950 | 2475 | 3400 | 24a | 8950 | 1 | 3 |
| 2S | 8350 | 2675 | 2010 | 25a | 3850 | 1 | 2 |
| 26 | 5850 | 855 | 1310 | 26a | 5400 | 1 | 1 |
| 27 | 3000 | 1520 | 1005 | 27a | 2300 | 1 | 4 |

### Spot Efficacy Test

For comparison, a Spot Efficacy test is employed with silver foil. In this test, 10 mg of 99.9% pure silver foil of 0.25 mm thickness is placed into a 15 cc sterile tube of capacity. Two milliliter of influent water that has 3.7×10⁵ CFU/ml E. coli. is added to tube. The tube having the influent water is shaken for one minute to produce treated influent water. One milliliter of the treated influent water is plated onto MacConkey agar that contains 5 g/L NaCl. The residual bacterial count, as measured by the spread plate method, is greater than 4000.

### Room Humidifier

In order to evaluate the effectiveness of the microbial control system in humidifiers, a microbial control system that includes a containment vessel having antimicrobial media therein is placed into the water tank of a humidifier such as a portable home humidifier. In this aspect, a model DF-1 “cool mist” humidifier from Duracraft is employed. The humidifier is rinsed thoroughly with ordinary tap water to remove any plasticisers or chemical residues that may be present prior to use.

The microbial control system includes a containment vessel which has antimicrobial media from Apyron Technologies, Inc. The containment vessel is formed from perforated polypropylene and has two nylon end caps. The containment vessel measures two inches long by one inch diameter with a capacity of 50 cc and a pore space of 30%.

The containment vessel is filled with 30 cc of 50:50 mix of MB2001-B and MB2002-B antimicrobial media from Apyron Technologies, Inc. The filled vessel is placed into the water tank of the humidifier (“Sample unit”). Chlorinated tap water from a sterile bottle is poured into the water tank of the sample unit.

For comparison, an identical model cool mist humidifier from Duracraft is employed except that the water tank of this humidifier lacks the filled containment vessel. (“Control unit”). Chlorinated tap water from a sterile bottle also is poured into the water tank of the Control unit.

Each unit is operated for 4-6 hours per day. At the end of a 4-6 hour period of operation, a 0.5 cc water sample is taken from the water tank of each unit by use of a sterile pipette. The water sample is deposited onto a sterile Fisher Scientific bacterial collection plate filled with Difco R2A Agar. A mist sample is gathered by holding a sterile Fisher Scientific bacteria collection plate in the mist path for two seconds. The samples on the collection plates are incubated for 5 days at room temperature. A Cell Counting Chamber from Bantex is used to count bacteria and fungi in each sample.

The units then sit overnight with residual water in place. The next day, the units are topped off with tap water, operated again for a period of 4-6 hours and sampled again. This procedure is repeated for 30 days. The results are shown in Tables 2 to 4.

TABLE 2

| Day No. | Control Mist CFU/ml | Sample Mist CFU/ml |
|---|---|---|
| 1 | 0 | 0 |
| 2 | 0 | 1 |
| 3 | 20 | 1 |

TABLE 3

| Week No. | Control Mist CFU/ml | Sample Mist CFU/ml |
|---|---|---|
| 1 | 11 | 0 |
| 2 | 750 | 1 |
| 3 | 6000 | 3 |

TABLE 4

| Week No. | Control Tank CFU/ml | Sample Tank CFU/ml |
|---|---|---|
| 1 | 400 | 2 |
| 2 | 600,000 | 200 |
| 3 | 1,000,000 | 20,000 |

Tables 2 and 3 show that during days 1-3 as well as during weeks 1-3 of humidifier use that the bacterial levels in the mist rise dramatically in the control unit. During these periods, however, the microbial control system controls the bacterial level in the mist in the sample unit. Table 4 shows that growth of bacteria in the water tanks occurs over a period of 21 days in both the control unit and the sample unit. The microbial control system is able to control the growth of bacteria in the tank water of the sample unit.

The microbial control system of the invention may also be used in other water treating systems such as cooling towers. Cooling towers are typically used in power plants or other industrial boiler systems to cool water that has been used for heat transfer. Such systems can contain over 100,000 gallons of water that is constantly being recycled. These boiler systems basically include a recirculating water supply in which the water is sent through piping that comes in contact with a heat source “condensers”. This water is then sent to a “cooling tower” where the heat is dissipated and the water is then returned to the condensers. This enables the water to be re-used many times. Traditionally, this water has been treated with caustic biocides and algicides to control the growth of various microorganisms.

When used in a cooling tower, the microbial control system, including a containment vessel and treatment media, is placed into the “cooling tower basin”. The water contacts the vessel and the antimicrobial treatment media whereby microorganisms in the water are controlled without the need to handle caustic biocidal liquids.

As an example, a 55 gallon containment vessel formed of stainless steel and having a pore space of 35% is filled with 250 pound of treatment media formed of a 50:50 mixture of 2-500 nm thick Ag on 2-3 mm alumina beads and 2-500 nm thick Cu on 2-3 mm alumina beads. The Ag is present in an amount of 0.7% based on total weight of Ag and alumina. Cu is present in an amount of 4.0% Cu based on total weight of Cu and alumina. The treatment media has a particle size of 5 mm. Water at a temperature of 40 C and at a flow rate of 1000 gallons per minute is flowed across the media in the container.

1. A microbial control system for control of microbial growth in water comprising antimicrobial treatment media within a containment vessel, the treatment media including any one or more of transition metals and transition metal oxides.

2. The system of claim 1 wherein the treatment media further comprises an inert support material.

3. The system of claim 2 wherein treatment media are in the form of any one of solid particles or layers transition metals or metal oxides on the support material.

4. The system of claim 3 wherein the transition metal is selected from the group consisting of Sc, Ti, V, Cr, Mn, Fe, Co, Ni, Cu, Zn, Y, Zr, Nb, Mo, Tc, Ru, Rh, Pd, Ag, Cd, Hf, Ta, W, Re, Os, Ir, Pt, Au, Hg, Rf, Db, Sg, Bh, Hs, Mt, Uun, Uuu and Uub.

5. The system of claim 3 wherein the transition metal is selected from the group consisting of Ag, Cu and Zn.

6. The system of claim 3 wherein the transition metal oxide is an oxide of any one of Ag, Cu, Zn and Sn.

7. The system of claim 3 wherein the transition metal oxide is an oxide of any one of Ag and Cu.

8. The system of claim 3 wherein the transition metal is a transition metal alloy of CuZn.

9. The system of claim 2 wherein the transition metal is Ag and the microbial control system provides solvated silver ions at a concentration of about 1 ppb to about 1000 ppb.

10. The system of claim 2 wherein the support material is selected from the group consisting of activated carbon, alumina, silica, titanium oxide, tin oxide, lanthanum oxide, copper oxide, vanadium oxide, manganese oxide, nickel oxide, iron oxide, zinc oxide, zirconium oxide, magnesium oxide thorium oxide, polyethylene, polypropylene, polyvinylchloride, polystyrene and polyethylene terephthalate.

11. The system of claim 2 wherein the support materials are selected from the group consisting of alumina and polyethylene terephthalate.

12. The system of claim 2wherein the treatment media has a metal content of about 0.01 wt. % to about 15 wt. %.

13. The system of claim 4 wherein the treatment media has a metal content of about 0.35 wt. % to about 3.5 wt. %.

14. The system of claim 3 wherein the treatment media is a mixture of Ag coated onto alumina and Cu coated on alumina wherein the Ag is present in an amount 0.7% Ag based on total weight of Ag and alumina and Cu is present in an amount of 4.0% Cu based on total weight of Cu and alumina.

15. The system of claim 2 wherein the treatment media are mixtures of nanoparticles of Ag and Cu wherein each of the Ag and Cu have a size of about 0.1 nm to about 10,000 nm.

16. The system of claim 2 wherein the treatment media is a mixture of nanoparticles of Ag and Cu wherein each of the Ag and Cu have a size of about 2 nm to about 500 nm and wherein the ratio of Ag to Cu in the mixture is about 1:1.

17. The system of claim 2 wherein the treatment media is a mixture of nanoparticles of silver and copper on alumina and wherein the silver nanoparticles have a median size of about 20 nm and the copper nanoparticles have a median size of about 100 nm.

18. The system of claim 17 wherein each of the silver nanoparticles and the copper nanoparticles are present in the mixture in an amount of about 0.2 wt. % to about 4.8 wt. % based on the total combined weight of the metal and the alumina support material.

19. The system of claim 17 wherein silver nanoparticles and the copper nanoparticles are present in the mixture in a ratio of 1:5.

20. The system of claim 2 wherein the treatment media comprises a mixture of silver oxide and copper oxide on alumna support material.

21. The system of claim 20 wherein the silver oxide is present in the mixture in an amount of about 0.1 wt. % to about 2 wt. %, remainder copper oxide.

22. The system of claim 3 wherein the treatment media is a mixture of nanoparticles of silver and copper in combination with nanoparticles of any one of additive metals or additive oxides wherein the additive metals are selected from the group consisting of Sc, Ti, V, Sn, Cr, Mn, Fe, Co, Ni, Zn, Y, Zr, Nb, Mo, Tc, Ru, Rh, Pd, Cd, Hf, Ta, W, Re, Os, Ir, Pt, Au, Hg, Rf, Db, Sg, Bh, Hs, Mt, Uun, Uuu and Uub
and wherein the additive metal oxides are selected from the group consisting of alumina, silica, silver oxide, titanium oxide, tin oxide, lanthanum oxide, copper oxide, vanadium oxide, manganese oxide, nickel oxide, iron oxide, zinc oxide, zirconium oxide, magnesium oxide, thorium oxide.

23. An ice making machine having an microbial growth control system for control of microbial growth in any of influent water and sump water processed by the ice making machine wherein the microbial control system comprises antimicrobial treatment media, the antimicrobial treatment media including any of transition metals or transition metal oxides, wherein the transition metal is selected from the group consisting of Sc, Ti, V, Cr, Mn, Fe, Co, Ni, Cu, Zn, Y, Zr, Nb, Mo, Tc, Ru, Rh, Pd, Ag, Cd, Hf, Ta, W, Re, Os, Ir, Pt, Au, Hg, Rf, Db, Sg, Bh, Hs, Mt, Uun, Uuu and Uub.

24. The machine of claim 23 wherein the influent water has a flow rate of more than about one bed volume per minute.

25. The machine of claim 24 wherein the influent water has more than about 5×10⁻⁶ m dissolved oxygen.

26. The machine of claim 25 wherein the influent water has a temperature of less than about 45 C.

27. The machine of claim 23 wherein the microbial control system includes a 50:50 mixture of component A formed from 2-500 nm thick Ag on 2-3 mm alumina beads and component B formed from 2-500 nm thick Cu on 2-3 mm alumina beads.

28. The machine of claim 27 wherein component A has 0.7% Ag based on total weight of Ag and alumina and component B has 4.0% Cu based on total weight of Cu and alumina.

29. The machine of claim 23 wherein the transition metal oxide is an oxide of any one of Ag and Cu.

30. The machine of claim 26 wherein the temperature of the influent water is about 45 C and the amount of dissolved oxygen is about 5×10⁻³ m to about 3×10⁻⁴ m.

31. A humidifier comprising a microbial control system for control of microbial growth in water processed by the humidifier, a microbial control system comprising antimicrobial treatment media, the antimicrobial treatment media including any of transition metals or transition metal oxides, wherein the transition metal is selected from the group consisting of Sc, Ti, V, Cr, Mn, Fe, Co, Ni, Cu, Zn, Y, Zr, Nb, Mo, Tc, Ru, Rh, Pd, Ag, Cd, Hf, Ta, W, Re, Os, Ir, Pt, Au, Hg, Rf, Db, Sg, Bh, Hs, Mt, Uun, Uuu and Uub.

32. The humidifier of claim 31 wherein the influent water has more than about 5×10⁻⁶ m dissolved oxygen.

33. The humidifier of claim 32 wherein the influent water has a temperature of less than about 35 C.

34. The humidifier of claim 33 wherein the microbial control system includes a 50:50 mixture of component A formed from 2-500 nm thick Ag on 2-3 mm alumina beads and component B formed from 2-500 nm thick Cu on 2-3 mm alumina beads.

35. The humidifier of claim 34 wherein component A has 0.7% Ag based on total weight of Ag and alumina and component B has 4.0% Cu based on total weight of Cu and alumina.

36. The humidifier of claim 31 wherein the transition metal oxide is an oxide of any one of Ag and Cu.

37. The humidifier of claim 35 wherein the amount of dissolved oxygen is about 5×10⁻³ m to about 3×10⁻⁴ m.

38. The humidifier of claim 31 wherein the humidifier is a mist type humidifier.

39. A cooling tower comprising a microbial control system for control of microbial growth in water processed by the cooling tower, the microbial control system comprising antimicrobial treatment media, the antimicrobial treatment media including any of transition metals or transition metal oxides, wherein the transition metal is selected from the group consisting of Sc, Ti, V, Cr, Mn, Fe, Co, Ni, Cu, Zn, Y, Zr, Nb, Mo, Tc, Ru, Rh, Pd, Ag, Cd, Hf, Ta, W, Re, Os, Ir, Pt, Au, Hg, Rf, Db, Sg, Bh, Hs, Mt, Uun, Uuu and Uub.
